(19) United States
(12) Patent Application Publication
(10) Pub. No.: US 2013/0273574 A1
(43) Pub. Date: Oct. 17, 2013

(54) OLIGOMERIC A-BETA IN THE DIAGNOSIS, PROGNOSIS, AND MONITORING OF ALZHEIMER'S DISEASE

(71) Applicant: Janssen Alzheimer Immunotherapy, Dublin (IE)

(72) Inventors: Daniel Kidd, Sunnyvale, CA (US); Johannes Rolf Streffer, Mortsel (BE)

(21) Appl. No.: 13/801,844

(22) Filed: Mar. 13, 2013

Related U.S. Application Data
(60) Provisional application No. 61/610,390, filed on Mar. 13, 2012.

(51) Int. Cl. G01N 33/68

(52) U.S. Cl. CPC G01N 33/6896
USPC 435/7.92; 436/501

(57) ABSTRACT

The invention provides methods for diagnosis, prognosis and monitoring of Alzheimer's disease. The methods involve measuring the amounts of combined monomeric and oligomeric Aβ and amount of monomeric Aβ in samples obtained from a subject, and determining a ratio. The ratio can be used in diagnosing, prognosing, and/or monitoring Alzheimer's disease.

### CROSS-REFERENCE TO RELATED APPLICATION

The present application is a non-provisional and claims the benefit of 61/610,390 filed Mar. 13, 2012, incorporated by reference in its entirety for all purposes.

### BACKGROUND

Alzheimer's disease (AD) is a progressive disease resulting in senile dementia (Selkoe, TINS 16:403 (1993); Hardy et al., WO 92/13069; Selkoe, J. Neuropathol. Exp. Neurol. 53:438 (1994); Duff et al., Nature 373:476 (1995); Games et al., Nature 373:523 (1995)). Broadly speaking, the disease falls into two categories: late onset, which occurs in old age (65+ years) and early onset, which develops well before the senile period, i.e., between ages 35 and 60. In both types of disease the pathology is the same, but the abnormalities tend to be more severe and widespread in cases beginning at an earlier age. AD is characterized by amyloid plaques, neurofibrillary tangles and cerebral neuronal loss. Neurofibrillary tangles are intracellular deposits of microtubule-associated Tau protein consisting of two filaments twisted about each other in pairs. Amyloid plaques are areas of disorganized neuropile up to 150 μm across with extracellular amyloid deposits at the center which are visible by microscopic analysis of sections of brain tissue. Early onset Alzheimer's is associated with genetic mutations in APP or presenilin genes, among others and trisomy of chromosome 21 in Down's syndrome.

The principal constituent of amyloid plaques is a peptide termed Aβ. Aβ is produced from the proteolytic processing of a large transmembrane glycoprotein, amyloid precursor protein (APP). The length of Aβ varies from 39 to 43 amino acids. The predominant form, Aβ40, is 40 amino acids in length and is considered a short form. The next most common form, Aβ42, is 42 amino acids in length and is considered a long form. Aβ42 is associated with pathogenicity and is the primary constituent in neuritic plaques (90%) and parenchymal vessel deposits (75%). Roher et al., Proc. Nat'l Acad. Sci. USA 90:10836 (1993). The carboxy terminus of Aβ includes part of the hydrophobic transmembrane domain of APP, which may account for its tendency to aggregate into the fibrils that form plaques.

Progressive cerebral deposition of Aβ can precede cognitive symptoms by years or even decades (Selkoe, J. Neuropath. and Exp. Neurol. 53:438 (1994) and Selkoe, Neuron 6:487 (1991)). Treatment and prophylaxis of AD would be facilitated by assays that detect the formation of amyloid plaques and/or other disease-associated physiological abnormalities prior to the onset of cognitive symptoms. Brain biopsies are highly intrusive and therefore undesirable, particularly in subjects not exhibiting cognitive symptoms. In vivo imaging of amyloid deposits has been reported as an alternative to brain biopsies (WO11/106732), but imaging techniques require complex and expensive equipment and specialized personnel to interpret the images.

Another approach is to detect biomarkers in tissue samples, particularly body fluids. Reduced levels of soluble Aβ42 have been reported in cerebral spinal fluid (CSF) of subjects with AD relative to normal controls. Another biomarker Tau, which is released by neuronal cell damage, has been reported as elevated in the CSF of AD patients (Vandermeeren et al., J. Neurochem. 61:1828 (1993)). Measurement of soluble Aβ and/or soluble Tau has been proposed for use in diagnosing and monitoring AD (see, e.g., U.S. Pat. No. 7,700,309). However, the ranges of these biomarkers in non-AD and AD subjects overlap, resulting in false negatives and positives.

### SUMMARY OF THE CLAIMED INVENTION

The invention provides methods of assisting in diagnosis, prognosis or monitoring of Alzheimer's disease or susceptibility thereto. Such methods comprise: (a) measuring an amount of monomeric Aβ in a sample of body fluid from a subject; (b) measuring an amount of monomeric and oligomeric Aβ in a second sample of body fluid from the subject; (c) comparing the amounts of monomeric Aβ and monomeric and oligomeric Aβ; and (d) using the comparison in the diagnosis, prognosis or monitoring of Alzheimer's disease or susceptibility thereto in the subject. Some methods determine a ratio between monomeric Aβ and monomeric and oligomeric Aβ, a lower quotient of monomeric Aβ over monomeric and oligomeric Aβ providing an indication of greater susceptibility to developing the disease, greater likelihood of presence of the disease, or deteriorating condition of the subject. Some methods determine a ratio between monomeric Aβ and oligomeric Aβ, a lower quotient of monomeric Aβ over oligomeric Aβ providing an indication of greater susceptibility to developing the disease, greater likelihood of presence of the disease, or deteriorating condition of the subject. Some methods determine an amount of oligomeric Aβ, a higher amount of oligomeric Aβ providing an indication of greater susceptibility to developing the disease, greater likelihood of presence of the disease, or deteriorating condition of the subject.

Some methods measure at least one of Aβx-37, Aβx-38, Aβx-39, Aβx-40, Aβx-41, and Aβx-42. Some methods measure at least Aβx-40. Some methods measure at least Aβx-42. Some methods measure at least Aβx-40 and Aβx-42.

In some methods, the amount of monomeric Aβ is measured using one or more antibodies that bind to one or more C-terminal epitopes present in monomeric Aβ and not present in oligomeric Aβ. In some methods, the one or more C-terminal antibodies are one or more end-specific antibodies for Aβ37, Aβ38, Aβ39, Aβ40, Aβ41, or Aβ42. In some methods, the one or more C-terminal antibodies include an antibody end-specific for Aβ40, optionally antibody 2G3. In some methods, the one or more C-terminal antibodies includes an antibody end-specific for Aβ42, optionally antibody 21F12. In some methods, the one or more C-terminal antibodies includes an antibody end specific for Aβ40 and an antibody end-specific for Aβ42. In some methods, the monomeric Aβ is measured by an immunoaffinity sandwich assay including the one or more C-terminal antibodies and another antibody that binds to an N-terminal and/or central epitope. In some methods, the other antibody binds to an N-terminal epitope, optionally antibody 3D6. In some methods, the other antibody binds to a central epitope, optionally antibody 266. In some methods, the one or more C-terminal antibodies are reporter antibodies and the other antibody is a capture antibody. In some methods, the one or more C-terminal antibodies are capture antibodies and the other antibody is a reporter antibody. In some methods, the one or more reporter antibodies are labeled with ruthenium and the capture antibody is labeled with biotin.

In some methods, measuring the amount of monomeric and oligomeric Aβ in step (b) comprises treating the sample with a disaggregating reagent that converts oligomeric Aβ to monomeric Aβ and determining the amount of monomeric Aβ in the disaggregating reagent-treated sample. In some methods, the disaggregating reagent comprises a chaotrope to solubilize oligomers into monomer. Chaotropes include: guanidine hydrochloride, guanidine isothiocyanate, urea, thiourea, lithium perchlorate, and/or potassium iodide. In some methods, the disaggregating reagent comprises a non-ionic detergent. In some methods, the disaggregating reagent comprises polyethylene glycol, polyvinylpyrolidone, a polyphenol, and/or certain small molecules, such as hexafluoroisopropanol. In some methods, the amount of monomeric Aβ in the disaggregating reagent-treated sample is measured by the same assay used to measure the amount of monomeric Aβ in step (a). In some methods, the measuring steps are performed by quantitative mass spectrometry. In some methods, the measuring steps are performed by capillary or gel electrophoresis, followed by quantitative western blotting.

In some methods, the body fluid sample is a CSF sample. In some methods, the body fluid sample is a blood sample. In some methods, the blood sample is a plasma sample. In some methods, steps (a) and (b) are performed simultaneously. In some methods, the sample of step (a) and the second sample of step (b) are different aliquots from a single sample.

In some methods, the subject does not have cognitive impairment and step (d) assesses the subject's susceptibility to developing Alzheimer's disease. In some methods, the subject has mild cognitive impairment and step (d) assesses the subject's susceptibility to developing Alzheimer's disease. In some methods, the subject has cognitive impairment and step (d) comprises using a combination of the comparison of step (c) and other symptom(s) and/or sign(s) of the subject’ condition to provide a diagnosis of Alzheimer's disease. In some methods, the subject has been diagnosed with Alzheimer's disease before performing the method, and step (d) provides an indication of stage of the disease. In some methods, the subject is receiving treatment or prophylaxis for Alzheimer's disease, and step (d) provides an indication of the subject's response to treatment. In some methods, the method is performed at intervals on the same subject and a change in the comparison in step (c) over time provides an indication of the subject's response to treatment.

In some methods, the subject is being treated with immunotherapy against Aβ. In some methods, the subject is being treated with bapineuzumab. Some methods further comprise treating the sample with an anti-idiotype antibody to bapineuzumab, optionally JH11.22G2, prior to performing steps (a) and (b). Some methods further comprise determining an amount of Tau or P-Tau in the sample, wherein increased Tau or P-Tau relative to a control value provides a further indication of susceptibility to developing Alzheimer's disease, presence of Alzheimer's disease, or deteriorating condition of the subject.

In some methods, the subject is a candidate for entry into a clinical trial to test a drug for treatment or prophylaxis of Alzheimer's disease, wherein if the quotient of monomeric Aβ over monomeric and oligomeric Aβ is below a threshold, the subject is included in the clinical trial, and if the subject is above the threshold the subject is excluded from the clinical trial. Some methods further comprise informing the subject or a care provider of the subject of the diagnosis, prognosis or monitoring.

In some methods, at least the step of comparing the amounts of monomeric Aβ and monomeric and oligomeric Aβ is implemented in a computer. In some methods, the computer receives signals relating to the amount of monomeric Aβ and the amount of monomeric and oligomeric Aβ, converts the signals to quantitative amounts, compares the quantitative amounts, and provides output relating to the amounts, comparison of the amounts, condition of the subject or recommended treatment of the subject.

The invention further provides methods of determining which subjects in a population to administer a drug to effect prophylaxis or treatment for Alzheimer's disease. Such methods comprise for each subject in the population: (a) measuring an amount of monomeric Aβ in a sample of body fluid; (b) measuring an amount of monomeric and oligomeric Aβ in a second sample of the body fluid; and (c) comparing the amounts of monomeric Aβ to monomeric and oligomeric Aβ, wherein subject(s) in the population receive or do not receive a drug to treat or effect prophylaxis for Alzheimer's disease based on the comparison. In some methods, the comparison determines a ratio between monomeric Aβ and monomeric and oligomeric Aβ, and subjects in which the quotient of monomeric Aβ over monomeric and oligomeric Aβ is below a threshold receive the drug.

The invention further provides methods of determining which treatment regime to administer to subjects in a population. Such methods entail measuring an amount of monomeric Aβ in a sample of body fluid; measuring an amount of monomeric and oligomeric Aβ in a second sample of the body fluid; and comparing the amounts of monomeric Aβ to monomeric and oligomeric Aβ. A first subpopulation of the subjects are treated with a first treatment regime and a second subpopulation of the subjects are treated with a second treatment regime wherein the ratio of monomeric to monomeric and oligomeric Aβ differs significantly between the subjects in the first and second subpopulations. In some such methods, the first treatment regime includes a drug for prophylaxis or treatment of Alzheimer's disease and the second treatment regime does not include the drug, and the subjects of the first subpopulation have a lower ratio of monomeric to monomeric and oligomeric Aβ than subjects of the second subpopulation. In some such methods, the quotient of monomeric Aβ over monomeric and oligomeric Aβ is below a threshold in subjects of the first subpopulation, and below a threshold in subjects of the second subpopulation. Measurement of Aβ forms and calculation of oligomeric Aβ-related parameters can be in accordance with any of the methods described herein.

The invention further provides methods of differentially treating subjects in a population, comprising treating a first subpopulation of the subjects with a first treatment regime and treating a second subpopulation of the subjects with a second treatment regime, wherein subjects in the first subpopulation and subjects in the second subpopulation have a significantly different average ratios of monomeric to monomeric and oligomeric Aβ. In some methods, subjects of the first subpopulation are treated with a drug for prophylaxis or treatment of Alzheimer's disease and subjects of the second subpopulation are not treated with the drug, and the ratio of monomeric to monomeric and oligomeric Aβ is significantly lower in the subjects of the first subpopulation than subjects in the second subpopulation.

The invention further provides methods of determining which subjects in population to enroll in a clinical trial, comprising for each subject in the population: measuring an amount of monomeric Aβ in a sample of body fluid; measuring an amount of monomeric and oligomeric Aβ in a second sample of the body fluid; and comparing the amounts of monomeric Aβ to monomeric and oligomeric Aβ, wherein subject(s) in the population are or are not enrolled in the clinical trial based on the comparison. In some methods, the comparison determines a ratio between monomeric Aβ and monomeric and oligomeric Aβ, and subjects in which the quotient of monomeric Aβ over monomeric and oligomeric Aβ falls below a threshold are enrolled in the clinical trial.

The invention further provides a diagnostic kit comprising: at least one C-terminal antibody end-specific for Aβ37, Aβ38, Aβ39, Aβ40, Aβ41, or Aβ42; an antibody binding to a N-terminal and/or central epitope of Aβ; and a disaggregating reagent that converts soluble oligomeric Aβ to monomeric Aβ. In some kits, the C-terminal antibody is end-specific for Aβ40 or Aβ42. Some kits comprise a C-terminal antibody end-specific for Aβ40 and a C-terminal antibody end-specific for Aβ42, providing multiple ratios for disease assessment to improve accuracy or sensitivity of the method result.

The invention further provides methods of screening an agent for activity against Alzheimer's disease, the method comprising: contacting a transgenic rodent model of Alzheimer's disease with the agent; comparing the amount of monomeric Aβ to the amount of monomeric and oligomeric Aβ in a body fluid of the transgenic rodent contacted with the agent; and using the comparison in determining whether the agent has activity useful in treating Alzheimer's disease.

The invention further provides methods of analyzing Aβ comprising: (a) measuring an amount of Aβ in a sample of body fluid from a subject, wherein the sample is not treated with a disaggregating reagent; (b) measuring an amount of Aβ in a sample of body fluid from the subject, wherein the sample is treated with a disaggregating reagent; and (c) comparing the amounts measured in steps (a) and (b). In some methods, the measuring in steps (a) and (b) is performed using an antibody that is end specific for a C-terminus of Aβ. In some methods, the comparison of step (c) determines a ratio of the amount in step (a) to the amount in step (b), or a difference between the amounts in step (a) and step (b). Some methods further comprise using the ratio or difference in the diagnosis, prognosis or monitoring of Alzheimer's disease or susceptibility thereto in the subject, a lower quotient of the amount in step (a) to the amount in step (b), or a higher difference between the amount in step (b) and the amount in step (a) providing an indication of greater susceptibility to developing the disease, greater likelihood of presence of the disease, or deteriorating condition of the subject.

In some methods, steps (a) and (b) measure at least one of Aβx-37, Aβx-38, Aβx-39, Aβx-40, Aβx-41, and Aβx-42. In some methods, steps (a) and (b) measure at least Aβx-40. In some methods, steps (a) and (b) measure at least Aβx-42. In some methods, steps (a) and (b) measure at least Aβx-40 and Aβx-42. In some methods, the amount of Aβ is measured using one or more C-terminal antibodies end-specific for Aβ37, Aβ38, Aβ39, Aβ40, Aβ41, or Aβ42. In some methods, the one or more C-terminal antibodies include an antibody end-specific for Aβ40 and an antibody end-specific for Aβ42. In some methods, Aβ is measured by an immunoaffinity sandwich assay including the one or more C-terminal antibodies and another antibody that binds to an N-terminal and/or central epitope. In some methods, the disaggregating reagent comprises guanidine hydrochloride, guanidine isothiocyanate, urea, thiourea, lithium perchlorate, and/or potassium iodide, a non-ionic detergent, polyethylene glycol, polyvinylpyrolidone, a polyphenol, and/or hexafluoroisopropanol. In some methods, steps (a) and (b) use the same assay to measure the amount of Aβ. In some methods, the body fluid sample is a CSF sample or a blood sample.

Some methods further include step (d) using the ratio or difference in the diagnosis, prognosis or monitoring of Alzheimer's disease or susceptibility thereto in the subject, a lower quotient of the amount in step (a) to the amount in step (b), or a higher difference between the amount in step (b) and step (a) providing an indication of greater susceptibility to developing the disease, greater likelihood of presence of the disease, or deteriorating condition of the subject. In some methods, the subject does not have cognitive impairment and step (d) assesses the subject's susceptibility to developing Alzheimer's disease. In some methods, the subject has mild cognitive impairment and (d) assesses the subject's susceptibility to developing Alzheimer's disease. In some methods, the subject has cognitive impairment and step (d) comprises using a combination of the comparison of step (c) and other symptom(s) and sign(s) of the subject’ condition to provide a diagnosis of Alzheimer's disease. In some methods, the subject has been diagnosed with Alzheimer's disease before performing the method and step (d) provides an indication of stage of the disease. In some methods, the subject is receiving treatment or prophylaxis for Alzheimer's disease, and step (d) provides an indication of the subject's response to treatment. In some methods, the method is performed at intervals and a change in the comparison in step (c) over time provides an indication of response to treatment. In some methods, the subject is being treated with immunotherapy against Aβ, such as bapineuzumab.

Some methods further comprise treating the sample with an anti-idiotype antibody to bapineuzumab, optionally JH11.22G2, prior to performing steps (a) and (b). Some methods, further comprise determining an amount of Tau or P-Tau in the sample, wherein increased Tau or P-Tau relative to a control value provides a further indication of susceptibility to developing Alzheimer's disease, presence of Alzheimer's disease, or deteriorating condition of the subject. Some methods further comprise informing the subject or a care provider of the subject of the diagnosis, prognosis or monitoring. Some such methods are performed on subjects in a population wherein a first subpopulation of the subjects are treated with a first treatment regime and a second subpopulation of the subjects are treated with a second treatment regime and the ratio of the amount of Aβ measured in step (a) to the amount of Aβ measured in step (b) is significantly lower in the subjects of the first subpopulation than the subjects of the second subpopulation. In some methods, the first treatment regime includes a drug for prophylaxis or treatment of Alzheimer's disease and the second treatment regime does not include the drug. In some methods, the ratio of the amount of Aβ measured in step (a) to the amount of Aβ measured in step (b) is below a threshold in the subjects of the first subpopulation and above the threshold in the subjects of the second subpopulation.

### DEFINITIONS

The term “antibody” includes intact antibodies and binding fragments thereof. Typically, fragments compete with the intact antibody from which they were derived for specific binding to an antigen. Fragments include separate heavy chains, light chains, Fab, Fab′, F(ab′)2, scFv, diabodies, Dabs, and nanobodies. Fragments are produced by recombinant DNA techniques, or by enzymatic or chemical separation of intact antibodies.

Specific binding refers to the binding of an antibody (or other agent) to a target (e.g., a component of a sample) that is detectably higher in magnitude and distinguishable from non-specific binding occurring to at least one unrelated target. Specific binding can be the result of formation of bonds between particular functional groups or particular spatial fit (e.g., lock and key type) whereas nonspecific binding is usually the result of van der Waals forces. Specific binding does not however imply that an agent binds one and only one target. Thus, an agent can and often does show specific binding of different strengths to several different targets and only nonspecific binding to other targets. Specific binding usually involves an association constant of 10⁷, 10⁸ or 10⁹ M⁻¹ or higher.

The term “epitope” refers to a site on an antigen to which an immunoglobulin or antibody (or antigen binding fragment thereof) specifically binds. Epitopes can be formed both from contiguous amino acids or noncontiguous amino acids juxtaposed by secondary and/or tertiary folding of a protein. Epitopes formed from contiguous amino acids are typically retained on exposure to denaturing solvents whereas epitopes formed by secondary and/or tertiary folding are typically lost on treatment with denaturing solvents. An epitope typically includes at least 3, 4, 5, 6, 7, 8, 9, 10, 11, 12, 13, 14 or 15 amino acids in a unique spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, G. E. Morris, Ed. (1996).

When an antibody is said to bind to an epitope within specified residues, such as Aβ 1-11, what is meant is that the antibody specifically binds to a polypeptide containing the specified residues (i.e., Aβ 1-11 in this an example). Such an antibody does not necessarily contact every residue within Aβ 1-11. Nor does every single amino acid substitution or deletion within Aβ 1-11 necessarily significantly affect binding affinity.

An end-specific antibody specifically binds to an epitope at the very N- or C-terminus an Aβ peptide (i.e., the epitope includes the N-terminal or C-terminal amino acid of the peptide) but binds less strongly or does not specifically bind to the residues constituting the epitope in a longer form of Aβ or in APP. Thus, an antibody that is end-specific for Aβ40 means that the antibody preferentially binds (e.g., association constant at least ten-fold higher) Aβ40 relative to an Aβ peptide ending at residue 37, 38, 39, 41, 42, or 43 Likewise, an antibody that is end-specific for Aβ42 means that the antibody preferentially binds an Aβ peptide ending at residue 42 over an Aβ peptide ending at residue 37, 38, 39, 40, 41, or 43.

The term “subject” includes human and other mammalian subjects. The term can refer to an individual anywhere on a spectrum from having no signs or symptoms of disease and to an individual with full symptoms of disease. Individuals in this spectrum can progress from being asymptomatic to having one or more signs of disease to one or more symptoms to full-blown disease. Signs and symptoms of disease can develop sequentially or concurrently. Individuals at any of these stages may or may not have genetic or other known risk of developing the disease.

Alzheimer's disease can be diagnosed by the criteria of DSM-IV-TR.

Mild Cognitive Impairment can be diagnosed by the 2001 guidelines of the American Academy of Neurology. In brief, these guidelines require an individual's report of his or her own memory problems, preferably confirmed by another person; measurable, greater-than-normal memory impairment detected with standard memory assessment tests; and normal general thinking and reasoning skills and ability to perform normal daily activities.

An individual is at elevated risk of Alzheimer's disease if the individual does not yet have the disease as conventionally defined (e.g., by DSM IV TR) but has one or more known risk factors (e.g., >70 years old, genetic, biochemical, family history, prodromal symptoms and/or oligomeric Aβ parameter as disclosed herein) placing the subject at significantly higher risk than the general population of developing the disease in a defined period, such as five years.

Susceptibility refers to the probability or risk of developing a disease and/or imminence of developing the disease. A higher probability or risk means a higher susceptibility. A shorter period between measurement and onset of disease also indicates higher susceptibility.

The term “symptom” refers to subjective evidence of a disease, such as altered gait, as perceived by the subject. A “sign” refers to objective evidence of a disease as observed by a physician.

Statistical significance implies a p value ≦0.05.

A diagnostic, prognostic or monitoring assay is usually less than 100% accurate in determining a present or future condition in a subject or changes therein but is nevertheless useful if the information resulting from the assay is indicative of a significantly greater or lesser likelihood of the presence or future development of the condition than would be the case without the information provided by the assay.

### DETAILED DESCRIPTION

### I. General

The invention provides methods for assisting in the diagnosis, prognosis and/or monitoring of Alzheimer's disease (AD) including progression to onset thereof. Although practice of the invention is not dependent on an understanding of mechanism, it is believed that oligomeric Aβ accounts for a substantial fraction of the soluble Aβ present in bodily fluids of AD patients and goes largely undetected by current immunoassay methods. Oligomeric Aβ is believed to be either a causative agent of cognitive symptoms in AD or an intermediate in the formation of amyloid plaques, themselves causative agents the manifestation of cognitive symptoms in AD. Failure to detect oligomeric Aβ in body fluids in previous reports may explain the significant overlap between values of Aβ in body fluids of subjects with and without Alzheimer's disease. The present methods can assess the oligomeric content of Aβ in body fluids and use this assessment in diagnosing AD, providing a prognosis for AD patients, and/or monitoring disease progression in AD patients. Such assessment is particularly useful for diagnosis and monitoring at early stages of the disease before a diagnosis of Alzheimer's disease can be made by conventional criteria.

### II. Aβ

Aβ is the principal component of amyloid plaques characteristic of Alzheimer's disease. Aβ has several naturally occurring full-length forms (resulting directly from cleavage of amyloid precursor protein (APP) by β and γ secretases without further degradation). The most common natural full-length forms of Aβ are referred to as Aβ39, Aβ40, Aβ41, Aβ42, and Aβ43. Exemplary sequences of these peptides and their relationship to APP, the large transmembrane glycoprotein from which they are derived, is illustrated in FIG. 1 of Hardy et al. TINS 20:155-158 (1997).

Aβ42 has the following sequence: NH₂-Asp Ala Glu Phe Arg His Asp Ser Gly Tyr Glu Val His His Gln Lys Leu Val Phe Phe Ala Glu Asp Val Gly Ser Asn Lys Gly Ala Ile Ile Gly Leu Met Val Gly Gly Val Val Ile Ala-COOH (SEQ ID NO: 1).

Natural forms Aβ41, Aβ40, Aβ39, Aβ38 and Aβ37 differ from Aβ 42 in that they lack the C-terminal Ala, Ile-Ala, and Val-Ile-Ala, Val-Val-Ile-Ala, Gly-Val-Val-Ile-Ala amino acid residues, respectively; Aβ43 differs from Aβ42 in that it includes an additional Thr amino acid residue at its C-terminus. Any of these forms can include a naturally occurring polymorphic variants of the above sequence such as the Arctic mutation. Truncated forms of Aβ are generated in vivo by additional degradation of Aβ (i.e., other than by β and γ secretases) or degradation in vitro after obtaining a sample of body fluid. Some naturally occurring Aβ fragments are N-terminally truncated. Examples of N-terminally truncated Aβ peptides identified to date include Aβ peptides having amino acid residues 6-42, 11-40, 11-43, 12-43, or 17-40. Other naturally occurring Aβ fragments identified to date feature truncations from both the N-terminus and C-terminus. Examples of such peptides include Aβ peptides having amino acid residues 3-34, 6-27, 6-34, 6-35, or 11-34. Other fragments of Aβ may result from degradation in isolated samples although any such degradation is preferably minimal.

Some techniques for measuring Aβ do not necessarily distinguish between full-length forms of Aβ and fragments thereof present in a body fluid sample. For example, an immunoassay with one antibody end-specific to the C-terminus of Aβ40 and another antibody specific to a central epitope of residues 20-25 can detect Aβ40 and Aβx-40 fragments, where x is from about 1-20. Thus, when Aβ is measured by such an assay, the assay actually measures Aβ40 and any fragments thereof having the form Aβx-40, wherein x is from about 1-20. Other assays measure essentially only full-length forms of Aβ. For example, an immune assay with one antibody end-specific to the C-terminus of Aβ40 and another antibody end-specific to the N-terminus (e.g., binding to or within an epitope of residues 1-5) detects Aβ40 without detecting subfragments (beyond a background or negative control level). Other assays, such as, quantitative mass spectrometry, can measure full-length forms of Aβ individually as well as fragments individually. Because some assays do not discriminate between full-length Aβ and certain fragments thereof, reference to Aβ includes full-length Aβ and fragments present in a body fluid sample under detection, unless the context, i.e., the nature of the assay requires otherwise. For brevity, the symbol Aβ x-y can be used to indicate Aβ peptides and any fragments thereof ending at residue y, where y can be 37, 38, 39, 40, 41, 42, or 43. For example, Aβ x-42 is used to indicate full-length Aβ42 or any fragment ending at residue 42 of the amino acid sequence provided above Likewise Aβ x-40 indicates full-length Aβ40 or any fragment thereof ending at residue 40.

Aβ peptides and fragments thereof exist in monomeric, oligomeric, protofibril and fibrillar forms representing different degrees of aggregation. Monomeric Aβ means Aβ that has the expected molecular weight of a monomer irrespective of presence or absence of a disaggregating solvent or reagent. The expected molecular weight of full-length forms of monomeric Aβ is about 3900 to 4700 Da depending on length (e.g., Aβ42 and Aβ40 have molecular weights of 4514 and 4330 Da respectively). Truncated forms have a proportionally smaller molecular weight depending on length. Molecular weight can be assessed on a gel, column (e.g., by HPLC), or mass spectrometer, among other approaches. Monomeric Aβ can also be recognized by lack of change in measured molecular weight on treatment with a disaggregating reagent. Monomeric Aβ can also be defined functionally as Aβ recognized by an antibody that exhibits at least ten fold higher preference for binding to a control preparation of monomeric Aβ over a control preparation of oligomeric Aβ, for example, an antibody end-specific for the C-terminus of a full-length form of Aβ, such as Aβ40 or Aβ42. A freshly dissolved preparation of Aβ in DMSO exists predominantly in monomeric form and provides a useful control to assess the molecular weight of a test preparation. A preparation of Aβ in water that has been allowed to stand for a several days and from which oligomeric Aβ has been isolated by gel electrophoresis or column chromatography, such as size exclusion chromatography or immunoaffinity chromatography that separates monomer from oligomer, can be used as a control for oligomeric Aβ.

Oligomeric Aβ means at least two molecules of Aβ non-covalently aggregated to one another. Oligomeric Aβ is believed to be held together at least in part, by hydrophobic residues at the C-terminus of the peptide (part of the transmembrane domain of APP). Like monomeric Aβ, oligomeric Aβ is soluble under physiological conditions. Most oligomeric Aβ has about 2-20 or 5-20 molecules of Aβ. Oligomeric Aβ can be recognized by the molecular weight of at least a dimeric molecule. For example, oligomers of full length Aβ have a molecular weight of at least about 7500 Da. Oligomers of Aβ fragments may have molecular weights less than 7500 Da, but most have molecular weights greater than 4600 Da. Oligomeric Aβ can also be recognized by a decrease in molecular weight on treatment with a disaggregating reagent. All or most oligomeric Aβ in body fluids can be converted to monomeric form having a molecular weight of no more than about 4600 Da by a disaggregating reagent. Under defined disaggregating conditions most or all oligomeric Aβ in body fluids is converted to monomeric form when there is no further change in molecular weight from continued treatment with the disaggregating reagent and/or there are no forms detectable having molecular weight characteristic of oligomers. Some epitopes, particularly C-terminal epitopes, recognized by antibodies to monomeric Aβ are not detectable in oligomeric Aβ. This may result from partial to total masking of the epitopes due to the physical associations between the individual Aβ peptides that make oligomeric Aβ, structural rearrangements in the individual Aβ peptides that make up oligomeric Aβ that destroy the epitopes, or a combination thereof. An amount of oligomeric Aβ can thus be functionally defined as the difference between (1) an amount of monomeric and oligomeric Aβ measured after treatment with a disaggregating agent and (2) an amount of monomeric Aβ measured without treatment with the disaggregating agent.

On gradual molecular rearrangement and further polymerization, oligomeric Aβ produces aggregates having greater than 20 Aβ peptides and an extended protofibrillar and then fibrillar structure. Unlike oligomeric Aβ, which is soluble under physiological conditions, fibrillar Aβ is typically insoluble under physiological conditions. Because of its insolubility, fibrillar Aβ is found in deposits, such as amyloid plaques. It has been proposed that plaques formed from fibrillar Aβ may be responsible for the cognitive defects associated with Alzheimer's disease. Alternatively or additionally oligomeric Aβ has been proposed as a causative agent in Alzheimer's disease. Regardless of whether oligomeric Aβ is a causative agent or an intermediate to a causative agent, its analysis in the present methods is a useful indicator for diagnosis, prognosis or monitoring.

Aβ40 and Aβ42 are the most common forms of Aβ found in humans. Aβ40 is about ten times more abundant than Aβ42 in the blood and CSF, but Aβ42 is the predominant form found in aggregated Aβ. For example, Roher et al. (Proc. Nat'l Acad. Sci. USA 90:10836 (1993)) found that Aβ42 represents 90% of the Aβ in neuritic plaques and 75% of the Aβ in parenchymal vessel deposits. In addition, Aβ42 has a greater propensity to form oligomers in solution and Aβ42 oligomers form fibrils significantly faster than Aβ40 oligomers. Bitan et al., Proc. Nat'l Acad. Sci USA 100:330 (2003). Because of their relative abundance, measurement of Aβ40 or Aβx-40 and/or Aβ42 or Aβx-42 in a bodily fluid such as blood, serum, plasma or CSF can be used as a surrogate for measurement of total soluble Aβ without individual detection of other forms of Aβ (e.g., Aβx-37, Aβx-38, Aβx-39, Aβx-41). However, the methods of the invention include measurement of any of the forms of Aβ (e.g., Aβx-37, Aβx-38, Aβx-39, Aβx-40, Aβx-41, Aβx-42), either alone or in combination. In measurements on the CSF, it is preferable to measure at least Aβ42 or Aβx-42. In measurement of blood, it is preferable to measure at least Aβ40 or Aβx-40.

### III. Measuring Monomeric and Oligomeric Aβ

The present methods can measure an amount of oligomeric Aβ in a body fluid. This measurement is preferably performed by measuring both a combined amount of oligomeric Aβ and monomeric Aβ and an amount of monomeric Aβ. Alternatively or additionally, the present methods can measure an amount of oligomeric Aβ directly. An amount can be measured in units of weight or binding signal, among other units. Arbitrary units of signal strength can be converted to weight by a calibration curve with known amounts of analyte.

A variety of techniques can be used for measuring a combined amount of monomeric and oligomeric Aβ, and an amount of monomeric Aβ. Preferred techniques include quantitative immunoaffinity assays, which use antibodies to detect the target antigen. Use of a combination of antibodies is preferred, such as in a sandwich-type immunoaffinity assay. Assays preferably include at least one capture antibody and at least one reporter antibody, the capture and reporter antibodies recognizing different epitopes on the same target molecule. Quantitative immunoaffinity assays, including sandwich assays, can be solid phase (e.g., ELISA or bead-based (e.g., Luminex® beads)) or liquid phase (e.g., elctrochemiluminescence). Quantitative immunoaffinity assays are generally described, e.g., in Antibodies: A Laboratory Manual, by Harlow and Lane, Cold Spring Harbor Press, Cold Spring Harbor, N.Y. (1988). Examples of ELISA sandwich assays used to detect Aβ in samples obtained from human subjects are described in WO 99/27944 and U.S. Pat. No. 7,700,309. Alternatively, monomeric and/or oligomeric Aβ can be detected and quantified using mass spectrometry or electrophoresis (e.g., capillary or gel electrophoresis) followed by quantitative Western blot, either technique optionally performed in combination with an immunoaffinity capture technique (e.g., immunoprecipitation) and/or protein purification techniques (e.g., precipitation and/or chromatography, such as HPLC). Mass spectrometry-based analysis of Aβ has been described, e.g., in Iurascu et al., Anal. Bioanal. Chem. 395:2509 (2009), Portelius et al., Acta Neuropathol. 120:185 (2010), and Wang et al., J. Biol. Chem. 271:31894 (1996).

For immunoaffinity-based measurements of an amount of monomeric Aβ in a sample, at least one antibody used in the assay should distinguish between monomeric and oligomeric Aβ. Suitable antibodies include those binding to an epitope in the C-terminal region of Aβ (i.e., amino acid residues 29-43), preferably an end-specific antibody for the C-terminus. Because of conformational changes that distinguish monomeric and oligomeric Aβ, as well as hidden peptide-peptide interfaces and steric hindrance related thereto, some epitopes present on monomeric Aβ are not present or are masked on oligomeric Aβ and antibodies specific to epitopes in the C-terminal region of monomeric Aβ generally do not bind to oligomeric Aβ. Such antibodies thus allow detection of essentially only monomeric Aβ in a sample that contains both monomeric and oligomeric Aβ. C-terminal end-specific antibodies can be, e.g., specific for Aβ37, Aβ38, Aβ39, Aβ40, Aβ41, or Aβ42. Preferred C-terminal end-specific antibodies include antibodies specific to the C-terminus of Aβ40 (e.g., monoclonal antibody 2G3) and antibodies specific to the C-terminus of Aβ42 (e.g., monoclonal antibody 21F12). Such C-terminal epitope specific antibodies can be used alone or in combination with one or more additional C-terminal epitope specific antibodies (e.g., one or more antibodies end-specific for Aβ37, Aβ38, Aβ39, Aβ40, Aβ41, Aβ42 or Aβ43) to bind Aβ in a sample.

In a sandwich assay, the antibody or antibodies distinguishing between monomeric and oligomeric forms can be the capture or reporter antibody, but preferably the capture antibody or antibodies. The other antibody or antibodies used in such a sandwich assay bind to an epitope on monomeric Aβ distinct from the epitope(s) bound by the discriminating antibody or antibodies. For simplicity, the discriminating antibody or antibodies are referred to as capture antibodies, and the antibody or antibodies binding to distinct epitopes are referred to as reporter antibodies (but the reverse specificities are also possible). For example, when a C-terminal epitope specific antibody is used to capture monomeric Aβ, a central epitope (within residues 12-28) specific antibody (e.g., monoclonal antibody 266) or an N-terminal epitope (i.e., within residues 1-11) specific antibody (e.g., monoclonal antibody 3D6 or 10D5) can be used as the reporter antibody. Use of an antibody binding to a central epitope allows detection of N-terminally truncated forms of Aβ, some or all of which may not be detected with an N-terminal reporter antibody.

For immunoaffinity-based measurements of a combined amount of monomeric and oligomeric Aβ in a sample, the sample can be treated with a reagent (e.g., a solvent) that disaggregates oligomeric Aβ into monomeric Aβ. The disaggregated sample is then diluted to lower the concentration of disaggregating reagent to a level tolerated by the immunoaffinity agents (i.e., capture and/or reporter antibodies). Antibody tolerance of the disaggregating reagent can be determined empirically. Any reagent that can disaggregate oligomeric Aβ without, on appropriate dilution, inhibiting antibody-based recognition of disaggregated monomeric Aβ can be used. Suitable disaggregating reagents include a chaotrope, a non-ionic detergent, a solubilizing agent or lipophilicity enhancing agent, or any combination thereof (e.g., a combination of a chaotrope and a detergent). Disaggregating reagents can be used individually or in any effective combination, at any effective ratio, for the intended purpose of converting oligomeric Aβ to monomeric. Suitable chaotropes include, for example, guanidine hydrochloride, guanidine isothiocyanate, urea, thiourea, lithium perchlorate, and potassium iodide. Suitable non-ionic detergents include Tween® series detergents, Triton® series detergents, and Brij® series detergents. Other solubilizing/lipophilicity enhancing agents include hexafluoroisopropanol and polymers (e.g., polymers of polyethylene glycol, polyvinylpyrolidone, polyphenols) which range in size from 10,000 to 50,000 Da.

A maximum disaggregating reagent concentration is dependent on both the tolerance of the immunoaffinity agents (i.e., capture and/or reporter antibodies) and the sensitivity of the method. Typically, dilution of a disaggregated sample by about 1:5 to about 1:40 (e.g., about 1:5 to about 1:20, or about 1:10) will ensure antibody tolerance of the disaggregating agent with minimal or no impact on the sensitivity of the method. Accordingly, if the maximum tolerable concentration of urea (or guanidine hydrochloride) in an immunoassay is determined to be 0.5M, and the disaggregated sample is going to be diluted 1:10 prior to the immunoassay, then the maximum concentration of disaggregating reagent permissible in the disaggregated sample is 5 M. Similar analysis can be performed for detergents, solubilizing/lipophilicity enhancing agents (e.g., polymers), and combinations of solvents/disaggregating reagents. For example, for polymers of about 10,000 to about 40,000 Da, a maximum concentration can be in the range of about 5% to about 10%.

The sample is treated with the disaggregating reagent such that all or essentially all of the Aβ in the sample is in the monomeric state (i.e., further treatment does not detectably increase the signal in the subsequent assay). The combined amount of monomeric and oligomeric Aβ in a disaggregating reagent-treated sample can be measured by immunoassay, preferably a sandwich assay. Because there is no need to distinguish between monomeric and oligomeric Aβ in a disaggregating reagent-treated sample (i.e., because there is essentially no oligomeric Aβ in disaggregating reagent-treated samples), any combination of antibodies to Aβ binding non-overlapping epitopes can be used as capture and reporter antibodies. However, for more direct comparability between assays, the same assay used to measure an amount of monomeric Aβ in a sample (i.e., a sample that has not been treated with disaggregating reagent) is also preferably used (best practice) to measure a combined amount of monomeric and oligomeric Aβ in the disaggregating reagent-treated sample. Thus, for example, if a sandwich assay featuring a C-terminal specific capture antibody and a central or N-terminal epitope specific reporter antibody is used to measure an amount of monomeric Aβ, it is preferable to use the same sandwich assay, including the same C-terminal epitope specific capture antibody and central or N-terminal epitope specific reporter antibody, to measure a combined amount of monomeric and oligomeric Aβ. If different assays are used to make the two measurements, the measured values can be normalized, as appropriate to compensate for different strengths of binding of antibodies, by reference to measurements of control samples with known concentrations of monomeric Aβ or monomeric and oligomeric Aβ.

Measurement of a combined amount of monomeric and oligomeric Aβ can also be achieved by simply summing separate measurements of monomeric Aβ (e.g., as described above) and oligomeric Aβ. For immunoaffinity-based measurements, this can be accomplished by measuring an amount of oligomeric Aβ in a sample of a bodily fluid obtained from a subject using an antibody that recognizes oligomeric Aβ but not monomeric Aβ. Antibodies specific for oligomeric Aβ that do not bind to monomeric Aβ have been described, e.g., in WO04/031400.

Depending on the format of the assay, the discrimination between monomeric Aβ and combined monomeric and oligomeric Aβ may not be absolute. In other words, an antibody that preferentially binds monomeric over oligomeric Aβ may not discriminate absolutely, or treatment with a disaggregating reagent may not convert 100% of the oligomeric Aβ to monomeric Aβ. Furthermore, some Aβ that is actually monomeric may be scored as oligomeric due to association with protein(s) or other macromolecules masking the epitope (e.g., C-terminal epitope) used to discriminate between monomeric and oligomeric Aβ. Notwithstanding such lack of complete precision, the measurement of a body fluid sample before and after treatment with a disaggregating reagent using an immunoassay including an antibody that preferentially binds monomeric Aβ over oligomeric (e.g., end-specific C-terminal antibody) can be treated as acceptable surrogates for measurement of monomeric Aβ and combined monomeric and oligomeric Aβ and subject to subsequent data analysis accordingly.

Viewed in a different way, the method can be performed by differential detection of Aβ in a body fluid sample with and without treatment with a disaggregating agent, without the need to characterize what is detected as being monomeric, oligomeric, monomeric associated with protein or otherwise. In such methods, an amount of Aβ is detected in a sample of body fluid from a subject wherein the sample has not been treated with a disaggregating agent, an amount of Aβ is detected in another sample of body fluid from the subject wherein the sample has been treated with a disaggregating agent, and the detected amounts of Aβ are compared. Detection is performed with an antibody an antibody that is end-specific for a C-terminus of Aβ or other antibody that preferentially binds monomeric over oligomeric Aβ. The comparison determines a ratio or difference between the amounts of Aβ measured with and without treatment with a disaggregating step. The ratio or difference can be used in diagnosis, prognosis or monitoring of Alzheimer's disease in similar fashion as the ratio or differences of monomeric to oligomeric Aβ. Thus, all the discussion of ratios, quotients or differences of oligomeric and monomeric Aβ and their measurement and interpretation and application to differential treatment regimes applies mutatis mutandis to ratios between amounts of Aβ measured in the presence and absence of a disaggregating reagent with an immunoassay employing antibody that preferentially binds monomeric Aβ over oligomeric Aβ. For example, a lower quotient of the amount without a dissociating reagent to the amount with a dissociating reagent or a higher difference between the amount with a dissociating reagent and without a dissociating reagent provide an indication of greater susceptibility to developing the disease, greater likelihood of presence of the disease, or deteriorating condition of the subject. As in other methods, tested populations of subjects can be stratified into first and second subpopulations based on the above-mentioned quotient or difference and the subpopulations subject to differential treatment regimes. For example, a subpopulation with a lower quotient or higher difference can be treated with a drug for prophylaxis or treatment of Alzheimer's disease and a subpopulation with a higher quotient or lower difference can be treated without the drug (including receiving no treatment).

Preferably, measurements of an amount of monomeric Aβ and a combined amount of monomeric and oligomeric Aβ are performed on the same sample, e.g., different aliquots of the same sample. However, the measurement can be performed on different samples provided that there is a sound basis for believing that the samples are essentially the same, such as when multiple samples are collected sequentially at essentially the same time and from essentially the same location on the same subject. The measurements of an amount of monomeric Aβ and a combined amount of monomeric and oligomeric Aβ can be performed at the same time or sequentially, preferably using the same reagents and instruments. For samples obtained from subjects receiving passive immunotherapy for Alzheimer's disease (i.e., receiving therapeutic antibodies specific to Aβ, such as bapineuzumab), the sample is optionally treated with an agent that neutralizes the therapeutic antibody (e.g., an anti-idiotype antibody, such as JH11.22G2, which neutralizes bapineuzumab) before performing an immunoassay. Alternatively, the assay can be performed using antibodies to the central and C-terminal regions as capture and reporter assays. Bapineuzumab does not interfere with such an assay because it binds to a site distal to central or C-terminal antibodies.

### IV. Antibodies Specific to Aβ

Antibodies used for detecting Aβ can be approximately classified as binding to N-terminal, central or C-terminal epitopes of Aβ. N-terminal epitopes are from residues 1-11, central epitopes from residues 12 to 28 and C-terminal epitopes from residue 29 to the C-terminus (e.g., residue 37, 38, 39, 40, 41, 42, or 43). Antibodies that bind to an epitope in the C-terminal region of Aβ include, e.g., antibodies 2G3, 21F12, and 369.2B. Antibodies that bind to an epitope in the central region of Aβ include, e.g., antibodies 266, 15C11, 2B1, 1C2, 4G8 and 9G8. Antibodies that bind to an epitope in the N-terminal region of Aβ include, e.g., antibodies, 12B4, 12A11, 6C6, 3A3, 2H3, 10D5 and 3D6.

2G3 is an mAb that specifically binds to a C-terminal epitope located in human Aβ, specifically at the C-terminus of Aβ40 (Johnson-Wood et al., PNAS Feb. 18, 1997 vol. 94, 1550-1555).

21F12 is an mAb that specifically binds to a C-terminal epitope located in human Aβ, specifically at the C-terminus of Aβ42 (Johnson-Wood et al., PNAS Feb. 18, 1997 vol. 94 no. 4 1550-1555).

369.2B is an mAb that specifically binds to a C-terminal epitope located in human Aβ, specifically at the C-terminus of Aβ42. The 369.2B antibody and variants thereof are described, e.g., in U.S. Pat. No. 5,786,180.

Numerous other antibodies end-specific for a C-terminal epitope on a form of human Aβ have been described in the scientific literature and/or are commercially available (see, e.g., Horikoshi et al., Biochem. Biophys. Res. Commun. 319, 733-7 (2004) referring to hybridoma 82E1, 1A10 and 1C3, the first of which is end-specific for Aβ40 and the second and third of which are specific for Aβ42; Iwatsubo et al., Neuron 13, 45-53 (1994); Barelli et al., Mol. Med. 3, 695-707 (1997); Levites et al., J. Clin. Invest. 116, 193-201 (2006); world wide web alzforum.org/res/com/ant; Novos, Biologicals, cat# NB300-225 (end-specific for Aβ40 and Autogen Bioclear cat# ABT109 (end-specific for Aβ42)).

266 is an mAb that specifically binds to a central epitope located in the human Aβ, specifically residues 16-24. The 266 antibody and variants thereof are described, e.g., in US 20050249725 and WO01/62801. A cell line producing the 266 monoclonal antibody was deposited with the ATCC on Jul. 20, 2004, under the terms of the Budapest Treaty and has deposit number PTA-6123.

15C11 is an mAb that specifically binds to a central epitope located in the human Aβ, specifically residues 19-22. The 15C11 antibody and variants thereof are described, e.g., in U.S. Pat. No. 7,625,560 and WO 2006/066049. A cell line producing the 15C11 monoclonal antibody was deposited with the ATCC on Dec. 13, 2005 under the terms of the Budapest Treaty and has deposit number PTA-7270.

2B1 is an mAb that specifically binds to a central epitope located in the human Aβ, specifically residues 19-23. The 2B1 antibody and variants thereof are described, e.g., in US 20060257396and WO 2006/066171. A cell line producing the 2B1 antibody was deposited on Nov. 1, 2005, with the ATCC under the terms of the Budapest Treaty and was assigned accession number PTA-7202.

1C2 is an mAb that specifically binds to a central epitope located in the human Aβ, specifically residues 16-23. The 1C2 antibody and variants thereof are described, e.g., in US 20060257396 and WO 2006/066171. A cell line producing the 1C2 antibody was deposited on Nov. 1, 2005, with the ATCC under the terms of the Budapest Treaty and was assigned accession number PTA-7199.

9G8 is an mAb that specifically binds to a central epitope located in the human, specifically residues 16-21. The 9G8 antibody and variants thereof are described, e.g., in U.S. Pat. No. 7,625,560 and WO 2006/066049. A cell line producing the 9G8 antibody was deposited on Nov. 1, 2005, with the ATCC under the terms of the Budapest Treaty and was assigned accession number PTA-7201.

4G8 is an mAb that specifically binds to a central epitope located in the human Aβ, specifically residues 17-24 (Covance SIG-39220).

12B4 is an mAb that specifically binds to an N-terminal epitope located in the human Aβ, specifically residues 3-7. The 12B4 antibody and variants thereof are described in US20040082762 and WO03/077858.

12A11 is an mAb that specifically binds to an N-terminal epitope located in the human Aβ, specifically residues 3-7. The 12A11 antibody and variants thereof are described, e.g., in US20050118651A1, US 20060198851, WO04/108895A2, and WO 2006/066089. A cell line producing the 12A11 monoclonal antibody was deposited with the ATCC on Dec. 13, 2005 under the terms of the Budapest Treaty and has deposit number PTA-7271.

6C6 is an mAb that specifically binds to an N-terminal epitope located in the human Aβ, specifically residues 3-7. The 6C6 antibody and variants thereof are described, e.g., in US 20060257396and WO 2006/066171. A cell line producing the antibody 6C6 was deposited on Nov. 1, 2005, with the ATCC under the terms of the Budapest Treaty and assigned accession number PTA-7200.

3A3 is an mAb that specifically binds to an N-terminal epitope located in the human Aβ, specifically residues 3-7. 2H3 is a mAb that specifically binds to an N-terminal epitope located in the human Aβ, specifically residues 2-7. The 3A3 and 2H3 antibodies and variants thereof are described, e.g., in US 20060257396 and WO 2006/066171. Cell lines producing the antibodies 2H3 and 3A3, having the ATCC accession numbers PTA-7267 and PTA-7269 respectively, were deposited on Dec. 13, 2005 under the terms of the Budapest Treaty.

3D6 is an mAb that specifically binds to an N-terminal epitope located in the human Aβ, specifically, residues 1-5. A cell line producing the 3D6 monoclonal antibody (RB96 3D6.32.2.4) was deposited with the American Type Culture Collection (ATCC), Manassas, Va. 20108, USA on Apr. 8, 2003 under the terms of the Budapest Treaty and has deposit number PTA-5130. 10D5 is an mAb that specifically binds to an N-terminal epitope located in the human Aβ, specifically residues 3-7. A cell line producing the 10D5 monoclonal antibody (RB44 10D5.19.21) was deposited with the ATCC on Apr. 8, 2003 under the terms of the Budapest Treaty and has deposit number PTA-5129. 3D6 and 10D5 antibodies and humanized and chimeric forms thereof are further described, e.g., in US 20030165496 and 20040087777 and WO02/088306, WO02/088307, WO02/46237 and WO04/080419. Additional humanized 3D6 antibodies are described in US 20060198851and WO 2006/066089.

Other antibodies useful for measuring an amount of monomeric and/or oligomeric Aβ in bodily fluids can be isolated de novo. The antibodies can be derived from the immunization of any suitable animal, including a rabbit, mouse, rat, guinea pig, hamster, goat, cow, and chicken. Alternatively, the antibodies can be produced by an in vitro selection method, such as phage display, or by immunizing transgenic mice, which allows for other types of antibodies, including human antibodies, or nanoantibodies.

The antibodies can be polyclonal, monoclonal, chimeric, or humanized. End-specific antibodies are made by immunizing with a short peptide (e.g., 4-8 amino acids) terminating with the end of Aβ for which specificity is desired. For example a peptide of Aβ 38-42 can serve as an immunogen to generate an end-specific antibody to Aβ42, Aβ 37-41 can serve as an immunogen to generate an end-specific antibody to Aβ41, Aβ 36-40 can serve as an immunogen to generate an end-specific antibody to Aβ40, Aβ 35-39 can serve as an immunogen to generate an end-specific antibody to Aβ39, Aβ 34-38 can serve as an immunogen to generate an end-specific antibody to Aβ38, or Aβ 33-37 can serve as an immunogen to generate an end-specific antibody to Aβ37. The short peptide is linked to a carrier to help elicit an immune response. Antibodies are screened for ability to preferentially bind the desired form of Aβ relative to longer forms of Aβ, APP, or segments thereof, including the amino acids of the immunogen as part of a longer protein without the free end against which end-specificity is desired. Polyclonal end-specific antibodies can be made by a similar immunization and removing antibodies lacking the desired specificity on an affinity column of a longer form of Aβ, APP, or segments thereof, including the amino acids of the immunogen without the free end against which end-specificity is desired. Suitable antibodies and fragments thereof can be recombinantly produced. In addition, other recombinant proteins which mimic the binding specificity of antibodies can be used (see, e.g., synbodies described by WO/2009/140039).

Antibodies specific for Aβ may be prepared with an immunogen comprising the desired target epitope, such as an epitope in the N-terminal region (i.e., amino acid residues 1-11), an epitope in the central region (i.e., amino acid residues 12-28), or an epitope in the C-terminal region (i.e., amino acid residues 29-43) of Aβ. A carrier molecule can be coupled to an immunogen, and used to prepare antisera or monoclonal antibodies by conventional techniques. Suitable immunogens usually have at least five contiguous residues within Aβ and may include more than six residues. Carrier molecules include serum albumin, keyhole limpet hemocyanin, or other suitable protein carriers, as generally described in Hudson and Hay, Practical Immunology, Blackwell Scientific Publications, Oxford, (1980), Chapter 1.3.

Antibodies can be modified or unmodified, depending on the measurement assay being used. For example, capture antibodies can be coupled to an affinity agent, such as biotin, avidin, or a short peptide (e.g., a his-tag). The affinity agent can then be linked to a solid substrate by means of a specific, high affinity interaction (e.g., the binding of biotin to avidin or streptavidin). The solid substrate can be, e.g., a bead or the surface of a well, and the high affinity interaction of the affinity agent can be used to attach the capture antibody to the solid substrate. Alternatively, a secondary antibody specific to a portion of the capture antibody (e.g., a constant region) can be absorbed to a solid substrate (e.g., plastic dish, bead) and used to attach the capture antibody to the solid substrate. Likewise, reporter antibodies can be modified to include a label or a secondary antibody specific to a portion of the reporter antibody (e.g., a constant region) can be used to provide the label. The label on the reporter antibody or secondary antibody can be, e.g., an enzyme (e.g., linked by a chemical linker or fused in-frame with the antibody), a fluorescent molecule, a chemiluminescent agent, a chromophore, a radioisotope, or any other chemical or agent that provides a quantifiable signal.

### V. Samples

The present methods measure a combined amount of monomeric and oligomeric Aβ, an amount of monomeric Aβ, and/or an amount of oligomeric Aβ directly in a sample obtained from a subject. The methods can involve obtaining a sample from the subject and/or processing the sample before performing the measurements. The subject is typically a human but can also be a mammal, such as rodent, preferably a mouse, e.g., a transgenic mouse that functions as a model of Alzheimer's disease. Body fluids include, for example, cerebrospinal fluid (CSF), blood, urine, and peritoneal fluid. Blood can mean whole blood as well as blood plasma or serum.

Sample preparation can include storage (e.g., at room temperature, at 4° C., or frozen) and/or shipping of the sample. Other processing can include, for example, centrifuging blood to obtain plasma or coagulating and centrifuging blood to obtain serum. Further sample preparation, if any, depends on the assay format used to measure amounts of monomeric and/or oligomeric Aβ, and can include biochemical steps such as protein precipitation and/or column chromatography. Whereas polystyrene collection tubes have been observed to bind Aβ, leading to loss of sample quality, polypropylene tubes do not exhibit similar Aβ-binding affinity and are preferred.

### VI. Use of Measurements of Aβ

Raw measurements of amounts of combined monomeric and oligomeric Aβ (or oligomeric Aβ) and monomeric Aβ can be processed to information useful in diagnosis, prognosis and monitoring of Alzheimer's disease. Usually the methods provide an amount of monomeric Aβ and a combined amount of monomeric and oligomeric Aβ in a body fluid. These amounts can be compared to provide several useful parameters of a subject's condition. Preferably, a ratio is determined between an amount of monomeric Aβ and a combined amount of monomeric and oligomeric Aβ. The ratio can be expressed as a quotient of monomeric Aβ over monomeric and oligomeric Aβ or vice versa (inverse quotient). Because the reverse quotient is the reciprocal of the quotient, determination of a ratio is considered to determine both the quotient and reverse quotient. The quotient of monomeric Aβ over monomeric and oligomeric Aβ is a measure of the fraction of total Aβ in the body fluid in monomeric form. Subtracting this fraction from 1 gives the fraction of oligomeric Aβ in total Aβ in body fluid. Quotients or fractions can also be expressed as percentages. The amounts can also be compared by subtracting the amount of monomeric Aβ from the amount of monomeric and oligomeric Aβ to give an amount of oligomeric Aβ. Alternatively, the amounts can be compared by determining a ratio between monomeric Aβ and oligomeric Aβ. The parameters determined by these comparisons are collectively referred to as oligomeric Aβ-related parameters.

The oligomeric Aβ-related parameters are used in diagnosis, prognosis or monitoring. Diagnosis, prognosis and monitoring need not be mutually exclusive, because the same parameter can be useful in a variety of ways as applied to a continuum of disease state and progressions. For example, a parameter can indicate a present condition of the subject (diagnosis) and a prediction of future condition (prognosis). A parameter can provide a present diagnosis and be one of a series of parameters used in monitoring. In general, an increased amount of oligomeric Aβ in a body fluid is associated with increased susceptibility to the disease, increased likelihood of presence of the disease, or deteriorating condition of a subject. Parameters in the form of a ratio, particularly a ratio between monomeric and monomeric and oligomeric Aβ in a body fluid, are preferred to reduce distortions due to differences in total content of Aβ in body fluids among subjects. An increased amount of oligomeric Aβ decreases the quotient of monomeric Aβ over monomeric and oligomeric Aβ. Thus, a decreased quotient of monomeric Aβ over monomeric and oligomeric Aβ is associated with increased risk of developing the disease, increased likelihood of presence of the disease or deteriorating condition of a subject. Likewise, increased oligomeric Aβ decreases the quotient of monomeric Aβ over oligomeric Aβ, and a decreased quotient is associated with increased risk of developing the disease, increased likelihood of presence of the disease or deteriorating condition of a subject. Conversely, increased oligomeric Aβ increases the quotient of oligomeric and monomeric Aβ over monomeric Aβ, or of oligomeric Aβ over monomeric Aβ; the increased quotient is associated with increased risk of developing the disease, increased likelihood of presence of the disease or deteriorating condition of a subject. The comparison of monomeric and oligomeric Aβ can be processed in other ways and similarly associated with increased or decreased risk of developing the disease, increased or decreased likelihood of presence of the disease, or improving or deteriorating condition of the subject.

The various parameters determined by comparison of measured values of monomeric and oligomeric Aβ can be compared with baseline values for assisting in the diagnosis, prognosis or monitoring of Alzheimer's disease. A baseline value can be the value of a parameter determined from a control group of subjects. The control group can be a negative control group or a positive control group. A suitable negative control group are individuals below 60 years old not having any known signs or symptoms of Alzheimer's disease or any known genetic risk thereof. A suitable positive control group are individuals diagnosed with Alzheimer's disease. Alternatively, a baseline value can be a value of a parameter previously obtained for the same subject.

In a negative control group of subjects, the baseline value for the quotient of monomeric Aβ to monomeric and oligomeric Aβ is expected to be about 1.0 (e.g., between about 0.90 and about 1.10). A quotient lower than the mean quotient in the negative control group provides an indication that a subject is at increased susceptibility to Alzheimer's disease, or has increased likelihood of presence of Alzheimer's disease. Differences between the quotient in a subject and the mean quotient in a population that are statistically significant with at least 95% confidence are particularly useful in forming a diagnosis or prognosis. However, lesser confidence intervals (e.g., between about 67 and 95% confidence) are also of value in flagging an individual as being at risk and initiating assays of other biomarkers or monitoring of the quotient with time. A quotient lower than a subject's previously determined baseline value (beyond experimental error preferably assessed with at least 95% confidence) provides an indication that the subject's condition is deteriorating.

Baselines of parameters (sometimes referred to as thresholds) can also be defined based on previous assays of test subjects. For example, a parameter, e.g., a quotient of monomeric over monomeric and oligomeric Aβ can be measured in a population of subjects free of symptomatic Alzheimer's disease and the population subsequently followed to determine which subjects develop Alzheimer's disease. A baseline or threshold can then be set in which subjects falling below the threshold have one outcome (e.g., develop Alzheimer's disease) and subjects above the threshold have another outcome (e.g., stay free of Alzheimer's disease) within a known margin of error. Subjects with a value of the parameter exactly at the threshold are usually allocated all to one outcome or the other depending on how the threshold is set, or can be scored as inconclusive. The probability of error and consequent potential for false positives and negatives can be controlled by the value at which the threshold is set. As another example, a threshold can be set to determine presence or absence of Alzheimer's disease by comparing values of parameters in populations known to have or not have Alzheimer's disease. Again, the precise value of the threshold can be set so as to keep the number of false positives and false negatives within a tolerable range. The tolerable range may be different for different health care practitioners, but preferably threshold values are selected so that the number of false positives and/or false negatives is less than 20%, less than 15%, less than 10%, or preferably less than 5%. Different threshold values can be used for different prognoses, diagnoses and monitoring. Preferably, baseline and threshold values are determined using the same assay format (e.g., the same type of assay and the same reagents, such as the specific capture and reporter antibodies used in an immunoaffinity sandwich assay) used to determine the ratio in test subjects. Likewise, baseline and threshold values are preferably determined using the same sample preparation techniques used to determine the ratio in test subjects.

Usually the parameters from comparing measured amounts of monomeric and oligomeric Aβ assist in providing prognostic, diagnostic or monitoring information in combination with others signs and symptoms of the subject, in particular assessment of the cognitive abilities of the subject and/or levels of other biomarkers. ADAS-CO 11, ADAS-CO 12, DAASD, CDR-SB, NTB, NPI, MMSE are well-known scales for assessing cognitive function. Other biomarkers include ^{[18F]}FDG, MRI markers (BBSI and VBSI), CSF biomarkers Aβ42, Tau, and/or P-Tau, and PET imaging of Aβ in the brain. The signs and symptoms of Alzheimer's disease, if any, in a subject can determine the goal of the analysis. For example, in an asymptomatic subject, the goal is usually to determine susceptibility to Alzheimer's disease and/or monitor progression toward disease, if any, moving forward. In a subject with cognitive impairment, the object can be to determine susceptibility to developing Alzheimer's disease and monitor progression toward the disease, but the object can also be to determine or exclude presence of Alzheimer's disease. In subjects already diagnosed with Alzheimer's disease by other criteria (e.g., DSM-IV-TR), the object can be to determine a stage of the disease, confirm the diagnosis or monitor future progression of the disease. In a subject being treated, the object can be to measure the response to treatment.

Thus, for example, in a subject showing no symptoms of cognitive decline, a quotient of an amount of monomeric Aβ over a combined amount of monomeric and oligomeric Aβ that is lower than a baseline value for negative control subjects (as defined above) provides an indication that the subject is at increased susceptibility to developing Alzheimer's disease relative to the control population. For the same subject, a quotient of an amount of monomeric Aβ over a combined amount of monomeric and oligomeric Aβ less than a previously determined quotient for the subject provides an indication of progression toward Alzheimer's disease.

For a subject showing symptoms of mild cognitive impairment (MCI), a quotient of an amount of monomeric Aβ over combined amount of monomeric and oligomeric Aβ that is lower than a particular baseline value for negative control subjects provides an indication the subject is at enhanced susceptibility of developing Alzheimer's disease. Mild cognitive impairment is itself a recognized condition, and can be a prodromal phase of Alzheimer's disease, but can also occur for other reasons. Accordingly, the lower quotient combined with the symptoms of MCI provides an indication of enhanced susceptibility of Alzheimer's disease compared to a subject that has MCI and a normal quotient or who has the same quotient without MCI. For a subject with MCI, a quotient that is lower than a previously determined quotient for the subject provides an indication of progression toward Alzheimer's disease.

For subjects showing symptoms of cognitive decline in general, whether or not classified as MCI, the decline may be associated with Alzheimer's disease or development thereof, or an unrelated dementia. In such an individual, a quotient of an amount of monomeric Aβ to a combined amount of monomeric and oligomeric Aβ that is lower than a baseline value for negative control subjects can be used, optionally in combination with other signs and symptoms of disease to diagnose or exclude Alzheimer's disease.

For subjects that have already been diagnosed with Alzheimer's disease, a quotient of an amount of monomeric Aβ to a combined amount of monomeric and oligomeric Aβ that is lower than a threshold can be used to stage the condition. For example, thresholds can be defined that correspond to particular stages of Alzheimer's (e.g., mild, moderate, late stage). A quotient lower than a previously determined quotient for the subject provides an indication that a subject's condition is deteriorating. Thus, the quotient can be used to monitor the condition of the subject. If a subject is receiving therapy for Alzheimer's disease (e.g., immunotherapy, such as bapineuzumab immunotherapy), the quotient can be used to monitor response to therapy. The change in quotient over time depends on the treatment agent. For immunotherapy, the treatment agent may cause an initial decrease in quotient in body fluids as Aβ deposits in the brain are solubilized and released to body fluids. However, in time, the quotient may increase as oligomeric Aβ is cleared from body fluids. In other agents, such as a small molecule that inhibits Aβ aggregation, the quotient may increase in response to successful treatment without a transient decrease.

The quotient of an amount of monomeric Aβ over a combined amount of monomeric and oligomeric Aβ is discussed for purposes of illustration, but any of the parameters mentioned previously can additionally or alternatively be used in similar manner. Of course, there are some superficial differences in the methodologies. For example, when using a quotient of monomeric Aβ and oligomeric Aβ over monomeric Aβ, values above (rather than below) a particular baseline or threshold value provide an indication that a subject has or is susceptible to developing an Aβ-related condition. A baseline value generated from a population of negative control subjects, can be expected to be about 1.0 (e.g., between about 0.95 and about 1.10). For an amount of oligomeric Aβ, a threshold value indicative of Alzheimer's disease, or susceptibility to such a condition can be about 0.3 ng/mL.

The amount of Tau or phosphorylated Tau (i.e., P-Tau) in a sample from a subject is a preferred biomarker that can be used in conjunction with parameters calculated from amounts of monomeric and oligomeric Aβ to assist in the diagnosis or prognosis of an Aβ-related condition or in monitoring an Aβ-related condition. Tau is a microtubule-associated protein found in neurofibrillary tangles in the brains of Alzheimer's disease patients (Goedert et al., Neuron 3:519-526 (1989); Goedert, TINS 16:460-465 (1993). Increased levels of Tau, and particularly P-Tau, in the CSF have been correlated with neuronal damage and Alzheimer's disease. For example, about 300 pg per milliliter of Tau in the CSF can be used as a threshold indicator of having Alzheimer's disease with an amount of Tau that exceeds or is equal to 300 pg/mL in the CSF indicates a greater likelihood that the subject has or is susceptible to developing an Aβ-related condition and an amount of Tau that is less than 300 pg/mL in the CSF indicates a greater likelihood that the subject does not have or is not susceptible to developing an Aβ-related condition (see U.S. Pat. No. 7,700,309).

Tau can be detected by, e.g., immunoassay. Useful detection techniques include, e.g., immunoaffinity sandwich assays involving a capture antibody and a labeled reporter antibody, both specific for Tau (see U.S. Pat. No. 7,700,309 and PCT/US11/033649). Antibodies against Tau are commercially available (e.g., from Sigma, St. Louis, Mo.), otherwise known (U.S. Pat. No. 7,700,309), or can be prepared by conventional methods.

The present methods may require obtaining or receiving a body fluid sample from a subject, performing an assay on the sample to measure an amount of one or more analytes (e.g., monomeric Aβ and monomeric plus oligomeric Aβ), data analysis of measured values to provide diagnostic, prognostic or monitoring information, and communication of the information to the subject, care giver or health care provider. In some methods, all steps are performed by one or more individuals in the same entity (e.g., medical practice, hospital, or health care organization). Alternatively, the methods can be performed by individuals from different entities working under contract or otherwise in collaboration. For example, individual(s) in one entity may order an assay and obtain a subject sample, and communicate information to a subject or care giver. Individual(s) in another organization may perform the assay and some or all of the data analysis.

### VII. Computer-Implementation

One or more steps of the methods (other than wet chemistry steps) can be performed in a suitably programmed computer. Calculation of one or more oligomeric Aβ-related parameters can be performed in such a computer. Raw data from measurement of any of the forms of Aβ (monomeric, momomeric plus oligomeric, treated with or without denaturing solvent) can be processed into a numerical value (e.g., amount or concentration) in a computer using for example, a calibration curve associating raw signals with numeric values stored in the computer. The computer can also be programmed to provide output of measured amounts of Aβ in any of the forms detected, values of oligomeric Aβ-related parameters, condition of the subject (e.g., diagnosis, prognosis, monitoring, disease progression, risk of developing Alzheimer's disease) and/or treatment options.

The invention can be implemented in hardware and/or software. For example, different aspects of the invention can be implemented in either client-side logic or server-side logic. The invention or components thereof can be embodied in a fixed media program component containing logic instructions and/or data that when loaded into an appropriately configured computing device cause that device to perform according to the invention. A fixed media containing logic instructions can be delivered to a viewer on a fixed media for physically loading into a viewer's computer or a fixed media containing logic instructions may reside on a remote server that a viewer accesses through a communication medium in order to download a program component.

Hardware can be a personal computer or any information appliance for interacting with a remote data application, for example, a digitally enabled television, cell phone, or personal digital assistant. Information residing in a main memory or auxiliary memory can be used to program such a system and can represent a disk-type optical or magnetic media, magnetic tape, solid state dynamic or static memory, or the like. For example, the invention may be embodied in whole or in part as software recorded on such fixed media. The various programs stored on the main memory can include a program to receive signals relating to measurements of the forms of Aβ (monomeric, monomeric plus oligomeric, treated with or without denaturing solvent), a program to process such signals into numerical values (e.g., amount or concentration), a program to calculate values of oligomeric Aβ-related parameters from these measured amounts, a program to interpret Aβ-related parameters in terms of subject condition, prognosis or treatment plan and the like. Such a program may work in part by comparing one or more calculated values of oligomeric Aβ-related parameters in a subject with a stored database of such values associated with subject conditions, prognosis or treatment plans. The computer memory may also store a program to provide output of measured amounts of Aβ in any of the forms detected, values of oligomeric Aβ-related parameters, condition of the subject (e.g., risk of developing Alzheimer's disease) and/or treatment options. Output can be provided for example on a display by saving to an additional storage device (e.g., ZIP disk, CD-R, DVD, floppy disk, flash memory card), and/or printing to hard copy, e.g., on paper). The result of the processing can be stored or displayed in whole or in part, as determined by the user.

### VIII. Clinical Trials

The oligomeric Aβ-related parameters determined above can be used in determining whether to enroll a subject in a clinical trial. The clinical trials can be for testing a drug potentially useful for prophylaxis or treatment of Alzheimer's disease. The drug can be, e.g., an antibody (e.g., an antibody specific for Aβ) or an immunogen designed to induce antibodies to Aβ.

The oligomeric Aβ-related parameter is compared to an appropriate threshold value. The appropriate threshold value depends on the oligomeric Aβ-related parameter used and the purpose of the trial. For example, the threshold can be selected to identify only subjects that have a strong likelihood of having Alzheimer's disease, or it can be selected to identify subjects that have enhanced susceptibility to Alzheimer's disease. Subjects in the population that have an oligomeric Aβ-related parameter that is above or below the threshold value are eligible to participate in the clinical trial. For example, subjects in the population that have a quotient of an amount of monomeric Aβ over a combined amount of monomeric and oligomeric Aβ that is below the appropriate threshold value are eligible to participate in the clinical trial, whereas subjects in the population that have a quotient above the threshold value are not eligible to participate in the clinical trial. Alternatively, subjects in the population that have an inverse quotient of oligomeric and monomeric Aβ over monomeric Aβ or an amount of oligomeric Aβ that is above the appropriate threshold value are eligible to participate in the clinical trial, whereas subjects in the population that have such a quotient or amount below the threshold value are not eligible to participate in the clinical trial. Use of an oligomeric Aβ-related parameter as a criterion for enrolling subjects in the trial results in a more uniform population with none or fewer individuals present who lack Alzheimer's disease or enhanced susceptibility to the disease and are unlikely to show a false-positive response to the treatment being tested.

### IX. Altered Treatment Regimes

The oligomeric Aβ-related parameters determined above can also be used in determining which subjects receive or do not receive a treatment regime. Such a parameter is compared with an appropriate threshold value. Based on the comparison, a subject can be administered a drug to effect prophylaxis or treatment for Alzheimer's disease. Alternatively, for a subject already receiving a drug for prophylaxis or treatment of an Aβ-related condition, the comparison can indicate that the dosage the subject is receiving should be increased, decreased, or eliminated in favor of a different drug.

For example, subjects not receiving any treatment or prophylaxis for Alzheimer's disease can be classified as having a quotient of monomeric Aβ over monomeric and oligomeric Aβ above or below a threshold value with subjects below the threshold thereafter receiving treatment or prophylaxis and subjects at or above the threshold not receiving treatment or prophylaxis. Subjects already receiving a drug for prophylaxis or treatment of Alzheimer's disease can be classified by whether a quotient of monomeric Aβ over combined monomeric and oligomeric Aβ is above or below a threshold value, with subjects above the threshold continuing to receive the drug and subjects below the threshold having an adjustment of drug dosage or being switched to a new drug for prophylaxis or treatment of Alzheimer's disease. Alternatively, for a subject that is already receiving a drug, the quotient can be compared to a baseline value corresponding to a quotient of the same parameters previously determined for the subject. If the quotient is lower than the baseline value, the dosage can be increased or the subject switched to a new drug for prophylaxis or treatment of Alzheimer's disease. If the quotient is higher than the baseline value, the subject's drug dosage can be reduced or left unchanged.

Other oligomeric Aβ-related parameters, such as the quotient of monomeric and oligomeric Aβ over monomeric Aβ (inverse quotient) or the amount of oligomeric Aβ, can also be used in the methods of altering the treatment regime for a subject that has an Aβ-related condition or is susceptible to developing an Aβ-related condition. The methods are analogous to those described above, except that when comparing the quotient or amount of oligomeric Aβ to a threshold value, a quotient or amount higher than the threshold or baseline value can lead to prescribing to the subject (e.g., a subject not receiving any treatment) a drug to effect prophylaxis or treatment of Alzheimer's disease, increasing the subject's drug dosage, or switching the subject to a new drug for prophylaxis or treatment of Alzheimer's disease. An inverse quotient or amount of oligomeric Aβ lower than the threshold or baseline value can lead to decreasing the subject's drug dosage or leaving the dosage unchanged.

Amounts of monomeric Aβ and monomeric and oligomeric Aβ and any of the oligomeric Aβ-related parameters determined from comparison of these values can also be used in determining which of two or more treatment regimes to administer to subjects in a population. An oligomeric Aβ-related parameter is used to stratify the population into first and second subpopulations in which the Aβ-related parameter has a statistically significant difference in between the populations. For example, the mean of the ratio of monomeric Aβ to monomeric and oligomeric Aβ in the first subpopulation differs from the mean of that ratio in the second population by a statistically significant margin. Subjects in the first subpopulation are treated with a first treatment regime and subjects in the second subpopulation are treated with a second treatment regime different from the first treatment regime. A treatment regime can be a null regime (i.e., subjects receive no treatment). Thus, for example, subjects in the first subpopulation can receive a drug for prophylaxis or treatment of Alzheimer's disease and subjects in the second subpopulation can receive nothing (or at least not the same drug as the subjects in the first subpopulation). Such a differential regime is indicated, for example, if the ratio of monomeric to monomeric and oligomeric forms of Aβ is lower in the subjects of the first subpopulation than the second subpopulation. Alternatively, subjects in the first subpopulation can receive a first drug for prophylaxis or treatment of Alzheimer's disease and subjects in the second subpopulation can receive a second such drug. Alternatively, subjects in the first and second subpopulations can receive different dosages, frequencies or routes of treatment with the same drug for treatment or prophylaxis of Alzheimer's disease. Some populations are stratified such that all subjects in one subpopulation have a value of an oligomeric Aβ-related parameter at or above a threshold and all subjects in another subpopulation have a value of the oligomeric Aβ-related parameter at or below the threshold. Here and elsewhere in the this application, subjects with a value of the parameter exactly at the threshold are usually allocated all to one subpopulation or the other depending on how the threshold is set, or can be scored as inconclusive and not included in either population. The number of subjects in the treated population and its subpopulations should be sufficient that one or more of the oligomeric Aβ-related parameters differs to a statistically significant extent between the subpopulations. For example, the methods can be applied to populations including at least 20, 50, 100, 1000 or 10,000 subjects.

The invention further provides methods of differentially treating subjects in subpopulations stratified as described above. Subjects in the different subpopulations can be differentially treated by receiving or not receiving the same drug for prophylaxis or treatment of Alzheimer's disease, by receiving different drugs for prophylaxis or treatment of Alzheimer's disease or by receiving different dosages, frequencies or routes of administration of the same drug for prophylaxis or treatment of Alzheimer's disease.

### X. Kits

This invention also provides kits for performing assays that aid in the diagnosis, prognosis, and monitoring of Alzheimer's disease. The kits can include two or more Aβ-specific antibodies useful for measuring an amount of monomeric Aβ in a sample, such as a bodily fluid obtained from a subject. The antibodies can be, e.g., useful for performing an immunoaffinity sandwich assay. Preferably, the kit includes at least one capture antibody specific to Aβ, and at least one reporter antibody specific to Aβ that is capable to binding to monomeric Aβ at the same time as the at least one capture antibody. Either the at least one capture antibody or the at least one reporter antibody is selected for specific binding to monomeric Aβ and inability to bind to oligomeric Aβ. For example, the at least one capture antibody can include an antibody that binds to a C-terminal epitope, and the at least one reporter antibody can include an antibody that binds to an N-terminal and/or central epitope. Alternatively, the at least one capture antibody can include an antibody that binds to an N-terminal and/or central epitope, and the at least one reporter antibody includes an antibody that binds to a C-terminal antibody. Suitable antibodies include any antibody described herein, including any fragments of such antibodies. Preferred C-terminal epitope specific antibodies include antibodies end-specific for Aβ40 (e.g., mAb 2G3) and antibodies end-specific for Aβ42 (e.g., mAb 21F12), but one or more antibodies end-specific for any or all of Aβ37, Aβ38, Aβ39, or Aβ41 can be included instead of or in addition to antibodies end specific for Aβ40, Aβ42. Preferred central epitope specific antibodies include antibodies specific for an epitope in amino acid residues 12-28 of Aβ (e.g., mAb 266). N-terminal epitope specific antibodies include antibodies specific for an epitope in amino acid residues 1-11 of Aβ, preferably an epitope in amino acid residues 3-7 of Aβ (e.g., mAb 10D5) or amino acid residues 1-5 of Aβ (e.g., mAb 3D6).

The capture antibodies in the kit are optionally conjugated to an affinity agent, such as biotin, avidin, or a peptide tag (e.g., his-tag). Alternatively, the kit can include a secondary antibody that specifically binds the capture antibody. The reporter antibodies in the kit are optionally conjugated to a label, e.g., an enzyme, a fluorescent molecule, a chemiluminescent agent, a chromophore, a radioisotope, or any other chemical or agent that provides a quantifiable signal. Alternatively, the kit can include a secondary antibody that specifically binds the reporter antibody and includes a suitable label.

The kits of the invention can further include disaggregating reagent (e.g., a solvent) suitable for disaggregating oligomeric Aβ. The disaggregating reagent can be, e.g., any disaggregating reagent described herein. The kits of the invention can also include agents for blocking therapeutic antibodies present in a sample from a subject. For example the blocking agent can be an anti-idiotype antibody, such as an anti-idiotype antibody specific to bapineuzumab (e.g., mAb JH11.22G2). The kits of the invention can also include an instruction for using the contents of the kit to perform a measurement described herein, e.g., a measurement of monomeric Aβ or a combined measurement of monomeric and oligomeric Aβ, or to determine an oligomeric Aβ-related parameter, such as a ratio described above.

### XI. Transgenic Animal Assays

Many animal models of Alzheimer's disease have been reported (see, e.g., WO 93/14200, U.S. Pat. Nos. 5,604,102, 5,387,742, and 6,717,031). Particularly useful animal models for Alzheimer's disease include mammalian models, more particularly rodent models, and in particular murine and hamster models. Such animal models can include a transgene which encodes and expresses human APP or a fragment thereof. The human APP transgene can include a mutation that promotes or hastens the development of Alzheimer's disease in the animal model. The mutation can, e.g., be associated with a hereditary form of Alzheimer's disease. For example, the Swedish mutation (i.e., asparagine⁵⁹⁵-leucine⁵⁹⁶) or a mutation at amino acid 717 of APP associated with the London or Indiana familial Alzheimer's disease mutations. Such mutations have been described, e.g., in U.S. Pat. Nos. 7,700,309 and 6,717,031. These models are useful for screening compounds for their ability to affect the course of Alzheimer's disease, both to ameliorate and aggravate the condition. Because Alzheimer's disease is characterized by a decrease in the amount of monomeric Aβ and an increase in the amount of oligomeric Aβ in bodily fluids, effective treatments for Alzheimer's disease change oligomeric Aβ-related parameters. For example, agents that hasten the progress of Alzheimer's disease tend to decrease the quotient of the amount of monomeric Aβ over the combined amount of monomeric and oligomeric Aβ in a sample. Conversely, agents that slow or halt the progress of Alzheimer's disease may tend to increase the quotient of the amount of monomeric Aβ over the combined amount of monomeric and oligomeric Aβ in a sample, although there may be a transient decrease before any increase. Such test compounds include antibodies or fragments thereof, proteins, small organic compounds, and the like.

The methods involve administering a test compound to a transgenic animal model of Alzheimer's disease and measuring monomeric Aβ and a combined amount of oligomeric Aβ and monomeric Aβ, measuring monomeric Aβ and measuring oligomeric Aβ, or simply measuring oligomeric Aβ in a body fluid sample from the animal; and determining one or more oligomeric Aβ-related parameters for the animal based on the measurements. Depending on the oligomeric Aβ-related parameter determined, an increase or decrease in the parameter statistic as compared to a relevant baseline value indicates that the test compound ameliorates or aggravates Alzheimer's disease. The baseline value can be determined from a group of control animals (e.g., a genetically similar or identical group of animals) that has not received the test compound.

For example, the methods can include measuring an amount of monomeric Aβ in a bodily fluid from the animal, measuring a combined amount of monomeric and oligomeric Aβ in the bodily fluid, determining a quotient of the measured amount of monomeric Aβ over the measured combined amount of monomeric and oligomeric Aβ for the bodily fluid, and comparing the quotient to an appropriate baseline value. If the quotient is higher than the baseline value, the test compound is identified as a drug potentially useful for prophylaxis or treatment of Alzheimer's disease. Alternatively, if the quotient is lower than the baseline value, the test compound is identified as a drug that exacerbates or hastens the progression of Alzheimer's disease.

Alternatively, the methods can include measuring an amount of monomeric Aβ in a bodily fluid from the animal, measuring a combined amount of monomeric and oligomeric Aβ in the bodily fluid, determining an inverse quotient of the combined measured amount of monomeric and oligomeric Aβ over the measured amount of monomeric Aβ for the bodily fluid, and comparing the inverse quotient to an appropriate baseline value. If the quotient is lower than the baseline value, the test compound is identified as a drug potentially useful for prophylaxis or treatment of Alzheimer's disease. Alternatively, if the quotient is higher than the baseline value, the test compound is identified as a drug that exacerbates or hastens the progression of Alzheimer's disease.

Alternatively, the methods can include measuring an amount of monomeric Aβ in a bodily fluid from the animal, measuring a combined amount of monomeric and oligomeric Aβ in the bodily fluid, determining the amount of oligomeric Aβ in the bodily fluid, and comparing the amount of oligomeric Aβ to an appropriate baseline value. If the amount is lower than the baseline value, the test compound is identified as a drug potentially useful for prophylaxis or treatment of Alzheimer's disease. Alternatively, if the amount is higher than the baseline value, the test compound is identified as a drug that exacerbates or hastens the progression of Alzheimer's disease. Such methods can also be performed by measuring an amount of oligomeric Aβ directly.

### XII. Variations

The same principles and strategy described above for Alzheimer's disease and Aβ can be used mutatis mutandis for other amyloidogenic diseases and their component peptides. In other words, a ratio of monomeric to oligomeric plus monomeric amyloidogenic peptide in a body fluid (or other related parameter as discussed above) is used to provide a diagnosis, prognosis or monitoring of a subject with a relatively low quotient of monomeric to oligomeric plus monomeric amyloidogenic peptide providing an indication of presence, or susceptibility to disease or deteriorating condition of a subject. Some examples of amyloidogenic diseases and their component peptides are: diabetes mellitus type 2, IAPP (Amylin); Parkinson's disease and other Lewy body diseases, alpha-synuclein; transmissible spongiform encephalopathy (e.g. bovine spongiform encephalopathy), PrPSc; Huntington's Disease, huntingtin; medullary carcinoma of the thyroid, calcitonin (ACa1); cardiac arrhythmias and isolated atrial amyloidosis, atrial natriuretic factor (AANF); atherosclerosis, apolipoprotein AI (AApoA1); reactive amyloidosis, familial Mediterranean fever, familial amyloid nephropathy with urticaria and deafness, and rheumatoid arthritis, serum amyloid A (AA); aortic medial amyloid, medin (AMed); prolactinomas, prolactin (APro); familial amyloid polyneuropathy, transthyretin (ATTR); hereditary non-neuropathic systemic amyloidosis, lysozyme (ALys); dialysis related amyloidosis, beta-2 microglobulin (Aβ2M); Finnish amyloidosis, gelsolin (AGe1); lattice corneal dystrophy, keratoepithelin (Aker); cerebral amyloid angiopathy (Icelandic type), cystatin (ACys); systemic AL amyloidosis or multiple myeloma, immunoglobulin light chain AL; sporadic inclusion body myositis, S-IBM; heavy chain amyloidosis associated with several immunocyte dyscrasias. Other examples of amyloidogenic diseases and their peptides are provided in Table 1 of U.S. Pat. No. 6,936,246.

Although the invention has been described in detail for purposes of clarity of understanding, certain modifications may be practiced within the scope of the appended claims. All publications and patent documents cited herein are incorporated by reference in their entirety for all purposes to the same extent as if each were so individually denoted. Unless otherwise apparent from the context, any step, feature, aspect, element or embodiment can be used in combination with any other.

1-61. (canceled)

62. A method of analyzing Aβ comprising,
a. measuring an amount of Aβ in a sample of body fluid from a subject, wherein the sample is not treated with a disaggregating agent;
b. measuring an amount of Aβ in a sample of body fluid from the subject, wherein the sample is treated with a disaggregating agent; and
c. comparing the amounts measured in steps (a) and (b).

63. The method of claim 62, wherein the measuring in steps (a) and (b) is performed using a C-terminal antibody end-specific for Aβ.

64. The method of claim 62, wherein the comparing determines a ratio of the amount in step (a) to the amount in step (b) or a difference between the amounts in step (a) and step (b) further comprising;
d. using the ratio or difference in the diagnosis, prognosis or monitoring of Alzheimer's disease or susceptibility thereto in the subject, a lower quotient of the amount in step (a) to the amount in step (b), or a higher difference between the amount in step (b) and step (a) providing an indication of greater susceptibility to developing the disease, greater likelihood of presence of the disease, or deteriorating condition of the subject.

65. (canceled)

66. The method of claim 62, wherein steps (a) and (b) measure at least one of Aβx-37, Aβx-38, Aβx-39, Aβx-40, Aβx-41, and Aβx-42.

67. The method of claim 62, wherein steps (a) and (b) measure at least Aβx-40.

68. The method of claim 62, wherein steps (a) and (b) measure at least Aβx-42.

69. The method of claim 62, wherein steps (a) and (b) measure at least Aβx-40 and Aβx-42.

70. The method of claim 62, wherein the amount of Aβ is measured using one or more C-terminal antibodies end-specific for Aβ37, Aβ38, Aβ39, Aβ40, Aβ41, or Aβ42.

71. The method of claim 70, wherein the one or more C-terminal antibodies include an antibody end specific for Aβ40 and an antibody end-specific for Aβ42.

72. The method of claim 70, wherein Aβ is measured by an immunoaffinity sandwich assay including the one or more C-terminal antibodies and another antibody that binds to an N-terminal and/or central epitope.

73. The method of claim 62, wherein the disaggregating reagent comprises guanidine hydrochloride, guanidine isothiocyanate, urea, thiourea, lithium perchlorate, and/or potassium iodide, a non-ionic detergent, polyethylene glycol, polyvinylpyrolidone, a polyphenol, and/or hexafluoroisopropanol.

74. The method of claim 62, wherein steps (a) and (b) use the same assay to measure the amount of Aβ.

75. The method of claim 62, wherein the body fluid sample is a CSF sample or a blood sample.

76. The method of claim 62, wherein the subject does not have cognitive impairment and step (d) assesses the subject's susceptibility to developing Alzheimer's disease.

77. The method of claim 62, wherein the subject has mild cognitive impairment and step (d) assesses the subject's susceptibility to developing Alzheimer's disease.

78. The method of claim 62, wherein the subject has cognitive impairment and step (d) comprises using a combination of the comparison of step (c) and other symptom(s) and/or sign(s) of the subject’ condition to provide a diagnosis of Alzheimer's disease.

79. The method of claim 62, wherein the subject has been diagnosed with Alzheimer's disease before performing the method and step (d) provides an indication of stage of the disease.

80. The method of claim 62, wherein the subject is receiving treatment or prophylaxis for Alzheimer's disease, and step (d) provides an indication of the subject's response to treatment.

81. The method of claim 80, wherein the method is performed at intervals and a change in the comparison in step (c) over time provides an indication of response to treatment.

82. The method of claim 80, wherein the subject is being treated with immunotherapy against Aβ.

83. The method of claim 82, wherein the subject is being treated with bapineuzumab.

84. The method of claim 83, further comprising treating the sample with an anti-idiotype antibody to bapineuzumab, optionally JH11.22G2, prior to performing steps (a) and (b).

85. The method of claim 62, further comprising determining an amount of Tau or P-Tau in the sample, wherein increased Tau or P-Tau relative to a control value provides a further indication of susceptibility to developing Alzheimer's disease, presence of Alzheimer's disease, or deteriorating condition of the subject.

86. The method of claim 62, further comprising informing the subject or a care provider of the subject of the diagnosis, prognosis or monitoring.

87. The method of claim 62 performed on subjects in a population wherein a first subpopulation of the subjects are treated with a first treatment regime and a second subpopulation of the subjects are treated with a second treatment regime and the ratio of the amount of Aβ measured in step (a) to the amount of Aβ measured in step (b) is significantly lower in the subjects of the first subpopulation than the subjects of the second subpopulation.

88. The method of claim 87, wherein the first treatment regime includes a drug for prophylaxis or treatment of Alzheimer's disease and the second treatment regime does not include the drug.

89. The method of claim 87, wherein the ratio of the amount of Aβ measured in step (a) to the amount of Aβ measured in step (b) is below a threshold in the subjects of the first subpopulation and above the threshold in the subjects of the second subpopulation.
